(19) United States
(12) Patent Application Publication
(10) Pub. No.: US 2026/0334968 A1
(43) Pub. Date: Oct. 29, 2026

(54) INTRAVASCULAR LITHOPLASTY BALLOON SYSTEMS AND DEVICES WITH STERILE, DISPOSABLE SINGLE-PIECE AND UNITARY CABLE, HANDLE, HUB, CATHETER AND BALLOON

(71) Applicant: Cardiovascular Systems, Inc., Santa Clara, CA (US)

(72) Inventors: John R. BALLARD, Waconia, MN (US); Jacob T. WILLIAMS, Plymouth, MN (US); Austin P. PETRONACK, Plymouth, MN (US)

(21) Appl. No.: 19/189,565

(22) Filed: Apr. 25, 2025

Related U.S. Application Data
(63) Continuation of application No. PCT/US2023/073648, filed on Sep. 7, 2023.
(60) Provisional application No. 63/384,288, filed on Nov. 18, 2022.

(51) Int. Cl. A61B 17/22; A61B 17/00

(52) U.S. Cl. CPC A61B 17/22022; A61B 17/00234; A61B 2017/00172; A61B 2017/00238; A61B 2017/00292; A61B 2017/00477; A61B 2017/00557; A61B 2017/22025

(57) ABSTRACT

Various embodiments of the systems, methods and devices are provided for breaking up calcified lesions in an anatomical conduit wherein exemplary systems generate an electrical arc between two spaced-apart electrodes disposed within a fluid-filled balloon. In some embodiments, a single-piece, unitary assembly comprising an interconnection cable, an actuating handle, a hub, a catheter and an angioplasty balloon is provided.

### CROSS-REFERENCE TO RELATED APPLICATIONS

This application is a continuation of International application number PCT/US2023/73648, filed Sep. 7, 2023, and entitled INTRAVASCULAR LITHOPLASTY BALLOON SYSTEMS AND DEVICES WITH STERILE, DISPOSABLE SINGLE-PIECE AND UNITARY CABLE, HANDLE, HUB, CATHETER AND BALLOON, which claims priority to provisional application No. 63/384,288, filed Nov. 18, 2022, and entitled INTRAVASCULAR LITHOPLASTY BALLOON SYSTEMS AND DEVICES WITH STERILE, DISPOSABLE SINGLE-PIECE AND UNITARY CABLE, HANDLE, HUB, CATHETER AND BALLOON, the entire contents of which are incorporated herein by reference for all purposes.

### STATEMENT REGARDING FEDERALLY SPONSORED RESEARCH OR DEVELOPMENT

None

### BACKGROUND OF THE INVENTION

### Field Of The Invention

The invention relates to systems, devices and methods for breaking up calcified lesions in an anatomical conduit. More specifically, an electrical arc is generated between two spaced-apart electrodes disposed within a fluid-filled member, creating pressure waves.

### Description of the Related Art

A variety of techniques and instruments have been developed for use in the removal or repair of tissue in arteries and similar body passageways, including removal and/or cracking of calcified lesions within the passageway and/or formed within the wall defining the passageway. A frequent objective of such techniques and instruments is the removal of atherosclerotic plaque in a patient's arteries. Atherosclerosis is characterized by the buildup of fatty deposits (atheromas) in the intimal layer (i.e., under the endothelium) of a patient's blood vessels. Very often over time what initially is deposited as relatively soft, cholesterol-rich atheromatous material hardens into a calcified atherosclerotic plaque, often within the vessel wall. Such atheromas restrict the flow of blood, cause the vessel to be less compliant than normal, and therefore often are referred to as stenotic lesions or stenoses, the blocking material being referred to as stenotic material. If left untreated, such stenoses can cause angina, hypertension, myocardial infarction, strokes and the like.

Angioplasty, or balloon angioplasty, is an endovascular procedure to treat by widening narrowed or obstructed arteries or veins, typically to treat arterial atherosclerosis. A collapsed balloon is typically passed through a pre-positioned catheter and over a guide wire into the narrowed occlusion and then inflated to a fixed pressure. The balloon forces expansion of the occlusion within the vessel and the surrounding muscular wall until the occlusion yields from the radial force applied by the expanding balloon, opening up the blood vessel with a lumen inner diameter that is similar to the native vessel in the occlusion area and, thereby, improving blood flow.

The angioplasty procedure presents some risks and complications, including but not limited to: arterial rupture or other damage to the vessel wall tissue from over-inflation of the balloon catheter, the use of an inappropriately large or stiff balloon, the presence of a calcified target vessel; and/or hematoma or pseudoaneurysm formation at the access site. Generally, the pressures produced by traditional balloon angioplasty systems is in the range of 10-15 atm, but pressures may at times be higher. As described above, the primary problem with known angioplasty systems and methods is that the occlusion yields over a relatively short time period at high stress and strain rate, often resulting in damage or dissection of the conduit, e.g., blood vessel, wall tissue.

Shockwave Medical, Inc., markets an alternative to traditional relatively high pressure balloon angioplasty. FIG. 2 illustrates one model of a known, prior art, lithotripsy system marketed by Shockwave Medical. As shown in FIG. 2, a pulse generator is illustrated and configured to generate voltage pulses. The voltage pulses travel along wire conductors that are disposed within the IVL connector cable with actuating handle that is connected with the pulse generator. The wire conductors continue distally through the catheter connector, hub and catheter shaft and arriving at emitters or spaced-apart electrode pairs located within an angioplasty balloon.

FIG. 2 illustrates the catheter connector, hub and catheter shaft of the lithotripsy system of FIG. 2 in more detail. As shown, the catheter connector comprises a connection port configured to connect with a connection receptacle located at the distal end of the IVL connector cable. The connection between the catheter connector's connection port and the IVL connector cable's connection receptacle is shown in FIG. 4, wherein the catheter connector's connection port is inserted into the connection receptacle of the catheter connector to comprise an electrical connection.

The known device of FIGS. 1-3 thus requires the following steps to prepare the device for therapeutic delivery, taken from Shockwave Intravascular Lithotripsy (IVL) System with the Shockwave S4 Peripheral Intravascular Lithotripsy (IVL) Catheter Instructions for Use (“IFU”) (See Shockwave IFU Portal at Instructions for Use|Shockwave Medical; https://discover.shockwavemedical.com/ifu).
1. Prepare the insertion site using standard sterile technique.
2. Achieve vascular access using physician's preferred methodology and location.
3. Place an appropriately sized and length introducer sheath.
4. Select a balloon catheter size based on the provided balloon compliance chart and reference vessel diameter. The largest diameter balloon should be used if 1:1 sizing is not available.
5. Remove the IVL catheter from the package.
6. Prepare the balloon using standard technique. Fill a 20 cc (mL) syringe with 5 cc (mL) of 50/50 saline/contrast medium. Attach syringe to inflation port on catheter hub. Pull vacuum at least 3 times, releasing vacuum to allow the fluid to replace the air in the catheter.
7. Fill indeflator device with 10 cc (mL) of 50/50 saline/contrast medium. Disconnect syringe and connect indeflator to inflation port of catheter hub ensuring no air is introduced to the system.
8. Flush the guidewire port with saline.
9. Remove the protection sheath from the catheter.
10. Wet the balloon and distal shaft with sterile saline in order to activate the hydrophilic coating.
11. Insert the IVL connector cvable into a sterile sleeve or probe cover. Note that as required in the IFU for the intravascular lithotripsy (IVL) connector cable, the sterile sleeve must cover the “catheter connection” that is the interconnection between complementary interconnects between the catheter connector and the distal end of the IVL connector cable, discussed herein.
12. Remove the cap from the proximal end and attach the IVL catheter's connector to the IVL connector cable. See FIGS. 3 and 4.
13. Attach the other side of same IV L connector cable to the IVL generator.

Further, this prior art device's instructions for use (IFU) state that “This device is intended for single (one) time use only. DO NOT re-sterilize and/or reuse”.

We note in this regard that step 11 supra provides for placement of a sterile sleeve or probe cover over the IVL connector cable and handle to allow for reuse of that component which, therefore is not single use. The remainder of the components downstream (on the balloon side) of the IVL connector cable are to be discarded after use.

It would be advantageous to provide, inter alia, an IVL lithotripsy system that does not require the extra step of locating a sterile sleeve or probe cover and then covering the IVL Connector Cable with that sleeve or cover before using the system.

Further in this regard, once the sterile sleeve or cover is placed, the operator must now actuate the pulse generator using the handle within the IVL Connector Cable component through the sterile sleeve or probe cover. The sterile sleeve or probe cover intervenes between the operator and the actuating handle and, therefore, must be transparent to allow visualization of the underlying control(s) on the handle. The sleeve is also movable relative to the handle and the slidable position or location of the sleeve relative to the handle is not a predictable interaction and can impact operational control.

In addition, several assembly steps are required using the known device of FIGS. 1-3 that may advantageously be avoided:
A. As noted above, the IVL connector cable must be placed into a sterile sleeve or cover; see step 11 above.
B. Because the IV L connector cable is a separate component, i.e., is not integrated with the remaining components, it must be provided with two interconnection means, one interconnect at a proximal end for connecting to the IVL pulse generator, and one interconnect at the distal end for interconnecting with a proximal end of the catheter connector.
C. The catheter connector's proximal end must have a complementary connector to allow interconnection with the distal interconnect of the IVL connector cable.
D. Thus, the known device of FIGS. 1-3 requires (1) a separate and reusable IVL connector cable with two interconnects; and (2) a separate disposable catheter connector with integrated hub, catheter and balloon.
E. If, during the procedure, the catheter with balloon is to be replaced, the sterile sleeve previously placed around the IVL connector cable must be moved proximally to uncover the interconnect between the IVL connector cable and the catheter connector.
F. The IVL connector cable and catheter connector may then be disconnected.
G. The catheter connector, hub, catheter and balloon may be removed after disconnection with the IVL connector cable.
H. A new balloon, catheter, hub and catheter connector are then introduced.
I. The proximal end of the catheter connector is then connected with the distal end of the IVL connector cable.
J. Finally, the sterile sleeve is slid distally over the IVL connector cable. The IFU for the connector cable requires that the sterile sleeve cover the interconnect between the IVL connector cable and the catheter connector.

SeeIFU for Shockwave IFU Portal (Intravascular Lithotripsy (IVL) Connector Cable at https://discover.shockwavemedical.com/ifu for IVL Catheter Replacement steps E-J recited above.

This prior art structure and related preparation and therapeutic methodology is cumbersome and, in the case of the sterile sleeve covering the actuation handle, requires the operator to look through the sleeve to identify the correct actuator and/or setting. An improvement is highly desirable.

Various embodiments of the present invention address these issues, among others, discussed above.

### BRIEF DESCRIPTION OF THE SEVERAL VIEWS OF THE DRAWINGS

These drawings are exemplary illustrations of certain embodiments and, as such, are not intended to limit the disclosure.

FIG. 1 illustrates a known system.

FIG. 2 illustrates one embodiment of a known system.

FIG. 3 illustrates one embodiment of a known system.

FIG. 4 illustrates a perspective view of one embodiment of a console of the present invention.

FIG. 5 illustrates a perspective view of one embodiment of an assembly of the present invention.

FIG. 6 illustrates a side view of one embodiment of the present invention.

FIG. 7 illustrates a perspective view of one embodiment of the present invention.

FIG. 8 illustrates a block diagram of one embodiment of the present invention.

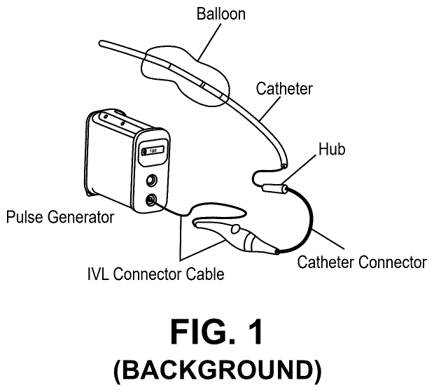

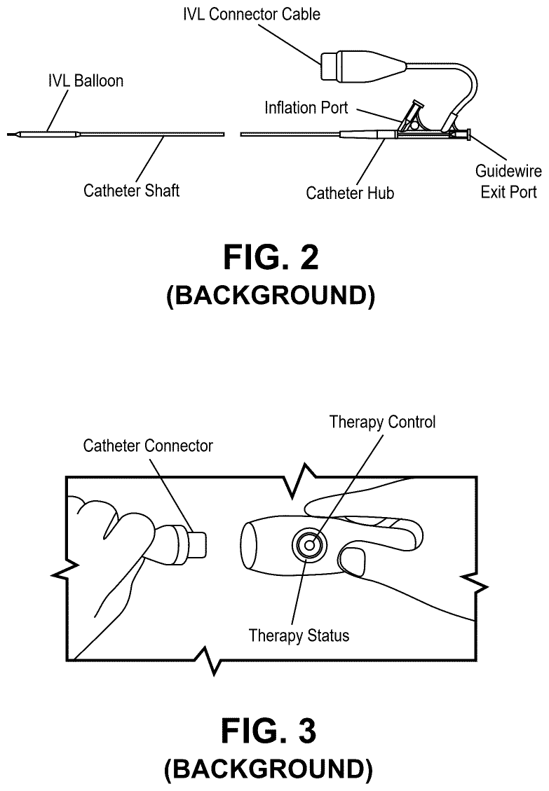

### DETAILED DESCRIPTION OF THE INVENTION

As discussed above, the prior art system currently marketed by Shockwave Medical, Inc., requires two interconnected components on the downstream or balloon side of the IVL pulse generator. One of the interconnected components, the IVL connector cable, requires a sterile sleeve or probe cover to be placed over the component before use to preserve sterility and allow the component to be reused. The second component, the catheter connector, hub, catheter and balloon is an assembly that is provided as an integrated, inseparable sterile unit and is single-use only.

The present invention avoids the multi-step assembly, and multi-step disassembly required if the catheter/balloon is to be replaced during therapy, of the prior art device.

In one embodiment, the entire portion of the disclosed system that is downstream of the pulse generator is integrated into an inseparable one-piece assembly. That is, the cable connecting with the IVL console, handle, hub, catheter shaft and balloon are all a sterile and single-piece, unitary structure with components that cannot be disconnected from each other or disassembled. This is in contrast to the prior art system discussed above that requires two separate structures downstream of the IVL pulse generator.

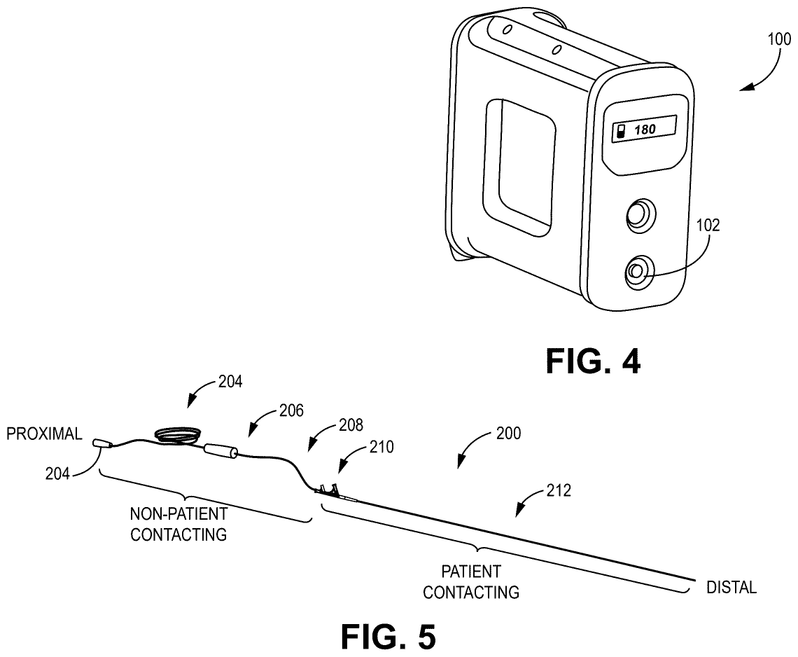

FIG. 4 illustrates an embodiment of the IVL pulse console or generator 100 that interconnects with the inventive assembly. A connector receptacle 102 for the single connection required for the inventive assembly is provided on the IVL pulse generator 100 as illustrated.

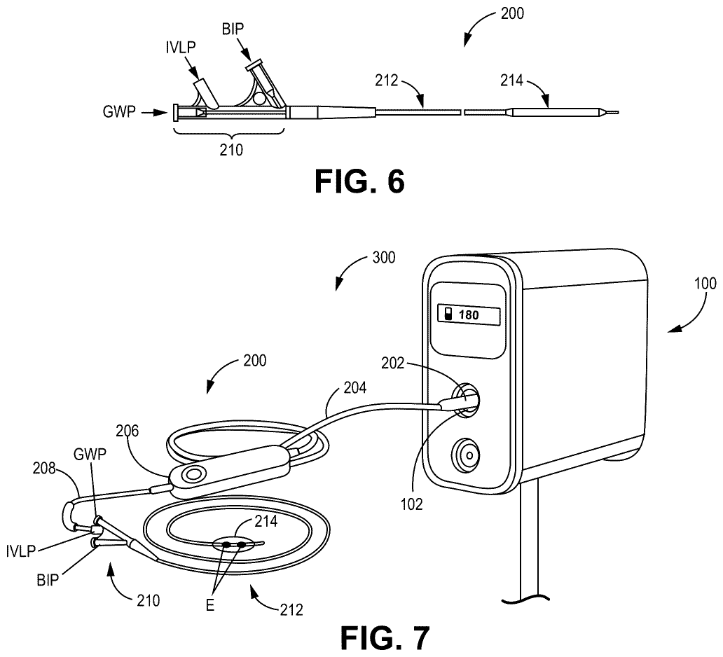

FIGS. 5 and 6 illustrate embodiments of the inventive assembly 200. In FIG. 5, moving from left to right (proximal to distal in the assembly 200), an interconnect 202 is provided at the proximal end of the assembly 200 which is configured to connect with the IVL pulse generator's connector receptacle 102 and which is, in turn, operatively connected with an electric cable 204 that leads to an actuating handle 206. The handle 206 is in operative connection with an IVL connector cable 208 which is, in turn, permanently connected with a portion of an access hub 210 (for allowing guidewire and inflation/deflation fluid access as well as operative electrical connection with wire conductors that are operatively electrically connected with the IVL pulse generator 100 via the connector receptacle 102) which operatively connects with a catheter 212. A fluid-inflatable balloon 214 is disposed at or near a distal end of the catheter shaft 212, wherein one or more pairs of spaced-apart electrodes are located within the balloon 214. Wire conductors run through or along the assembly between the IVL pulse generator 100 and at least one of the spaced-apart electrodes. Note that the entire assembly 200 is one-piece, and requires a single connector/connection between the interconnect 202 and the connector receptacle 102 to prepare the assembly for therapy.

FIG. 6 illustrates a side cutaway view of the assembly 200 of FIG. 5, from the hub 210 and showing an exemplary balloon 214 placement. The hub 210 comprises, as shown, a guide wire port GWP configured to allow guide wire access through the hub 210, catheter shaft 212 and balloon 214. The hub 210 further comprises a balloon inflation port BIP configured to allow fluid from a fluid reservoir (not shown) into the assembly 200 for inflation of the balloon 214. The balloon inflation port BIP is further configured to allow a pathway to remove fluid, or deflate, the balloon 214. The hub 210 further comprises an IVL connection port IVLP which is a permanent physical and operative electrical connection between the IVL connector cable 208. The IVLP is configured to provide electrical connection and communication from the pulse generator 100 to wire conductors disposed within the catheter shaft 212 which lead to the spaced-apart electrode pairs within the balloon 214.

FIG. 7 provides an embodiment of an assembled IVL system 300 comprising the single interconnection between the inventive single-piece and disposable assembly 200 described above and the IVL pulse generator 100, also described above.

Accordingly, in the embodiment of FIG. 7, the one-piece, unitary structure requires just one interconnection to set up power and control of the system 300, that is, the interconnection between the electrical cable 204 proximal to the actuating handle 206 and the IVL pulse generator 100. This is in contrast to the prior art system which requires two separate interconnections: (1) the interconnection between the proximal end of the IVL connector cable and the IVL pulse generator; (2) the interconnection between the distal end of the IVL connector cable and the proximal end of the catheter connector.

Moreover, the one-piece, unitary structure comprising the disposable catheter assembly 200 is entirely disposable, making preparation of the IVL system 300 for therapy simpler than the prior art system. In addition, if the inventive device's catheter/balloon 214, or other component of the disposable catheter assembly 200, requires replacement during a therapy, the entire assembly 200 is simply disconnected from the IVL pulse generator 100, removed from the patient, with a new assembly 200 located within the patient and then reconnected to the IVL pulse generator 100.

This is in contrast with the prior art system which, in order to preserve the IVL Connector Cable for reuse following a procedure, requires placement of a sterile sleeve or probe cover over the actuating handle.

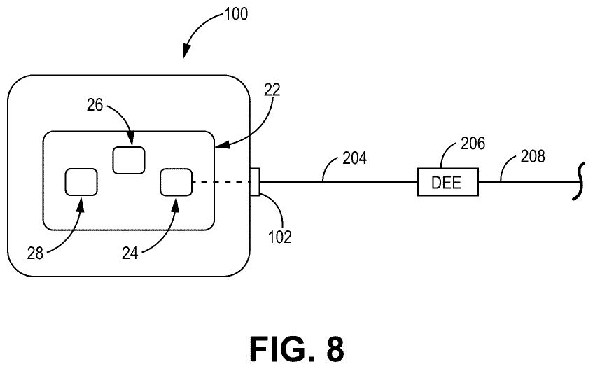

Turning now to FIG. 8, and with continued reference to FIG. 7, the actuating handle 206 may comprise a data encoding element DEE such as an EPROM or RFID or other similar device comprising a non-volatile memory and/or a device comprising a one-time programmable memory (OTP). The IVL pulse generator 100 may comprise an IVL control system comprising a processor 24 configured to execute instructions that may be stored on the memory 26 and further comprising circuitry 28 for IVL operations according to the processor 24 guidance. The data encoding element DEE may be in electrical communication with at least the processor 24.

Data encoding element DEE may comprise at least some, and in preferred embodiments, all, of the operational parameters relating specifically to the disposable catheter assembly 200. This configuration allows the IVL pulse generator 100 to conduct the IVL procedure according to the processor 24 guidance that may be modified depending on the specific catheter assembly 200 characteristics and features which are provided to the processor 24 by the data encoding element DEE. For example, IVL systems comprising different outer diameter sizes of balloons may require different voltage levels. Encoding this data into the data encoding element DEE enables the processor 24 to recognize the balloon size when the disposable catheter assembly 200 is connected to the IVL pulse generator as described above. Moreover, the data encoding element DEE may comprise a memory that may log or track certain events and communicate those logged or tracked events to the processor 24 for comparison against predetermined values and/or thresholds. Certain embodiments of the DEE's memory may comprise storage of therapeutic parameters that may be specific to the specific disposable catheter assembly 200 and that may be uniquely varied across different models of the disposable catheter assembly. The memory of the DEE may be in communication with the IVL console or generator 100 via connector receptacle 102.

Alternatively, the processor 24 may log or track certain events and store them in the associated memory 28 for comparison against predetermined values and/or thresholds. Still more alternatively, the data encoding element DEE and the processor 24 may each log or track certain events and store them for comparison against predetermined values and/or thresholds. In some embodiments, the data encoding element DEE may log or track certain events and provide that data to the processor 24 for comparison against predetermined values and/or thresholds. In certain embodiments, the tracked or logged events and/or other predetermined data stored within memory 28 may be communicated to the IVL console or generator 100 via connector receptacle 102. The ability to store the algorithm parameters, and any logged or tracked events discussed herein, within the data encoding element DEE allows console 100 algorithm work to be executed without having to hardcode on the console the specific therapeutic parameters and/or algorithm that is specific for a certain model of disposable catheter assembly 200. A related advantage is that any updates for existing catheter assemblies 200 and/or new therapeutic parameters and/or algorithms for new catheter assembly models 200 are stored on the data encoding element DEE and does not require releasing a new console 100 or updating a console 100 memory. Instead, the data encoding element DEE is simply connected with the console 100 and the required data is shared with, transmitted to, and/or accessed by the console 100.

In addition to storage of therapy parameters that are specific to the particular catheter assembly 200 that is connected to the IVL pulse generator 100, the data encoding element DEE and/or processor 24 may further comprise additional data, including but not limited to the following:
Logging of relevant events prior to, during and after execution of the therapy.
Tracking when the particular disposable catheter assembly 200 device was first connected to the IVL pulse generator 200, including in some embodiments tracking of subsequent re-connection events.
Tracking the number of voltage pulses generated and/or delivered for the particular connected disposable catheter assembly 200 device.
Logging of any faults or error conditions sensed or encountered during execution of the therapy.

In addition to provision of specific disposable catheter assembly 200 characteristics as described above, the data encoding element DEE enables easier investigation of device use history and can stop delivery of voltage pulses when the specific number of allowed voltage pulses provided data encoding element DEE have been generated.

The data encoding element DEE may further comprise a number of parameters that are specific to the disposable catheter assembly 200, including but not limited to:
Serial number
Model number
Number of total allowed voltage pulses
Maximum time allowed for voltage pulses after first connection with the IVL pulse generator
Catheter parameters and characteristics
Voltage pulse width time
Voltage pulse frequency (Hz)
Starting voltage set point
Ending voltage
Voltage step size
Number of voltage pulses applied prior to applying voltage step
Number of voltage pulses applied prior to a momentary pause in therapy enforced by the generator
Duration of momentary pause in therapy enforced by the generator
Catheter inflation profiles
Nominal pressure of the balloon
Diameter of the catheter's inflation lumen
Outer diameter of the balloon
Balloon compliance (D=f(P)
Balloon capacitance dV/dP
Balloon time constant D=D(P)*(1−e−bt)
Inflation rate
Inflation volume
Inflation time
Deflation time
Inflation velocity
Deflation velocity

If the console or pulse generator 100 detects a fault condition (either retrospective or prospective), wherein the console or pulse generator 100 determines for example and without limitation, that a voltage pulse did not generate an effective therapy and/or detects a voltage pulse that is determined to have been issued following device damage detection, the console or pulse generator 100 may write the fault detection occurrences to the data encoding element DEE therapy log and thereafter prevent further voltage pulses being generated to the connected catheter assembly 200. This may also coincide with an error message displayed to the user.

A non-exhaustive list of fault conditions that may be detected retrospectively (after delivery of a voltage pulse) comprises:
Pulses delivered if the catheter assembly 200 balloon is not inflated to a predetermined therapeutic pressure. This fault condition may indicate and/or include operator error or misuse or device damage such as a burst balloon.
Pulses delivered through the catheter assembly 200 with insufficient electrical integrity, for example, insulation broken down along conductor, proximal to an emitter.
Console or pulse generator 100 attempted to deliver a voltage pulse and did not detect a rise in current over a predetermined peak current threshold, indicating an unsuccessful pulse was generated and delivered.
A non-exhaustive list of fault conditions that may be detected prospectively or before generating and delivering a voltage pulse comprises:
The catheter assembly 200 attempted to be used to generate and deliver voltage pulses after a predetermined time period has passed. For example, no further voltage pulses maybe generated and delivered to a catheter assembly 200 after the catheter assembly 200 has been connected with the console or pulse generator 100 for more than a predetermined amount of time, e.g., and without limitation this predetermined connection threshold or limit may comprise 1-8 hours.
The catheter assembly 200 may be attempted to generate and deliver voltage pulses after the catheter assembly 200 and/or the console or voltage generator has/have reached a predetermined shelf life.
The catheter assembly may be attempted to generate and deliver voltage pulses after the maximum number of pulses for a catheter assembly have been generated and delivered.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1-15. (canceled)

16. An IVL catheter assembly, comprising:
an electrical cable comprising an interconnect at a proximal end of the electrical cable, wherein the interconnect is configured to connect to a connector receptacle of an IVL pulse generator;
an actuating handle located at a distal end of the electrical cable, wherein the actuating handle comprises a data encoding element comprising a memory storing one or more operational parameters associated with the IVL catheter assembly, wherein the data encoding element is configured for communicating the one or more operational parameters to the IVL pulse generator when the interconnect is connected to the connector receptacle;
an access hub connected to the actuating handle and connected to a catheter; and
a fluid-inflatable balloon located at or near a distal end of the catheter, wherein one or more pairs of spaced-apart electrodes are disposed within the fluid-inflatable balloon, wherein the IVL pulse generator is configured to generate one or more electrical arcs at the one or more pairs of spaced-apart electrodes when the interconnect is connected to the connector receptacle, and wherein the IVL catheter assembly comprises a sterile single-piece disposable catheter assembly.

17. The IVL catheter assembly of claim 16, wherein an IVL connector cable electrically connects the actuating handle to the one or more pairs of spaced-apart electrodes.

18. The IVL catheter assembly of claim 16, wherein the access hub is configured to facilitate guidewire access and inflation/deflation fluid access to the catheter.

19. The IVL catheter assembly of claim 16, wherein the one or more operational parameters comprise one or more catheter parameters and/or one or more balloon parameters.

20. The IVL catheter assembly of claim 16, wherein the one or more operational parameters comprise one or more of: a number of total allowed voltage pulses, a maximum time allowed for voltage pulse delivery after connection of the interconnect to the connector receptacle, a voltage pulse width time, a voltage pulse frequency, a starting voltage set point, an ending voltage, a voltage step size, or a number of voltage pulses to be applied prior to applying a voltage step.

21. The IVL catheter assembly of claim 16, wherein the one or more operational parameters comprise one or more of: a number of voltage pulses to be applied prior to enforcing a momentary pause in therapy or a duration of the momentary pause in therapy.

22. The IVL catheter assembly of claim 16, wherein the data encoding element is configured for logging event data associated with execution of an IVL therapy via the IVL catheter assembly when the interconnect is connected to the connector receptacle.

23. The IVL catheter assembly of claim 22, wherein the data encoding element is configured for communicating the event data to the IVL pulse generator when the interconnect is connected to the connector receptacle.

24. An IVL catheter assembly, comprising:
an electrical cable comprising an interconnect at a proximal end of the electrical cable, wherein the interconnect is configured to connect to a connector receptacle of an IVL pulse generator;
an actuating handle located at a distal end of the electrical cable, wherein the actuating handle comprises a data encoding element comprising a memory, wherein the data encoding element is configured for logging event data associated with execution of an IVL therapy via the IVL catheter assembly when the interconnect is connected to the connector receptacle;
an access hub connected to the actuating handle and connected to a catheter; and
a fluid-inflatable balloon located at or near a distal end of the catheter, wherein one or more pairs of spaced-apart electrodes are disposed within the fluid-inflatable balloon, wherein the IVL pulse generator is configured to generate one or more electrical arcs at the one or more pairs of spaced-apart electrodes when the interconnect is connected to the connector receptacle, and wherein the IVL catheter assembly comprises a sterile single-piece disposable catheter assembly.

25. The IVL catheter assembly of claim 24, wherein the event data comprises connection of the interconnect to the connector receptacle.

26. The IVL catheter assembly of claim 24, wherein the event data comprises a number of voltage pulses delivered to the one or more pairs of spaced-apart electrodes when the interconnect is connected to the connector receptacle.

27. The IVL catheter assembly of claim 24, wherein the event data comprises one or more detected fault conditions.

28. The IVL catheter assembly of claim 27, wherein the one or more detected fault conditions comprise one or more prospective faults or one or more retrospective faults.

29. The IVL catheter assembly of claim 24, wherein the data encoding element is configured for communicating the event data to the IVL pulse generator when the interconnect is connected to the connector receptacle.

30. The IVL catheter assembly of claim 24, wherein the memory of the data encoding element stores one or more operational parameters associated with the IVL catheter assembly, and wherein the data encoding element is configured for communicating the one or more operational parameters to the IVL pulse generator when the interconnect is connected to the connector receptacle.

31. An IVL system, comprising:
an IVL pulse generator comprising a control system and a connector receptacle; and
an IVL catheter assembly, comprising:
an electrical cable comprising an interconnect at a proximal end of the electrical cable, wherein the interconnect is configured to connect to the connector receptacle of the IVL pulse generator;
an actuating handle located at a distal end of the electrical cable, wherein the actuating handle comprises a data encoding element comprising a memory, wherein the data encoding element is configured for logging event data associated with execution of an IVL therapy via the IVL catheter assembly when the interconnect is connected to the connector receptacle;
an access hub connected to the actuating handle and connected to a catheter; and
a fluid-inflatable balloon located at or near a distal end of the catheter, wherein one or more pairs of spaced-apart electrodes are disposed within the fluid-inflatable balloon, wherein the IVL pulse generator is configured to generate one or more electrical arcs at the one or more pairs of spaced-apart electrodes when the interconnect is connected to the connector receptacle, and wherein the IVL catheter assembly comprises a sterile single-piece disposable catheter assembly.

32. The IVL system of claim 31, wherein the data encoding element is configured for communicating the event data to the control system of the IVL pulse generator when the interconnect is connected to the connector receptacle.

33. The IVL system of claim 32, wherein the control system is configured to disable voltage pulse generation to the one or more pairs of spaced-apart electrodes based on the event data.

34. The IVL system of claim 33, wherein the event data comprises one or more detected fault conditions.

35. The IVL system of claim 34, wherein the one or more detected fault conditions comprise one or more prospective faults or one or more retrospective faults.

36. An IVL catheter assembly, comprising:
an electrical cable comprising an interconnect at a proximal end of the electrical cable, wherein the interconnect is configured to connect to a connector receptacle of an IVL pulse generator;
a data encoding element comprising a memory storing one or more operational parameters associated with the IVL catheter assembly, wherein the data encoding element is configured for communicating the one or more operational parameters to the IVL pulse generator via the electrical cable when the interconnect is connected to the connector receptacle;
an actuating handle located at a distal end of the electrical cable;
an access hub connected to the actuating handle and connected to a catheter; and
a fluid-inflatable balloon located at or near a distal end of the catheter, wherein one or more pairs of spaced-apart electrodes are disposed within the fluid-inflatable balloon, wherein the IVL pulse generator is configured to generate one or more electrical arcs at the one or more pairs of spaced-apart electrodes when the interconnect is connected to the connector receptacle, and wherein the IVL catheter assembly comprises a sterile single-piece disposable catheter assembly.

37. The IVL catheter assembly of claim 36, wherein the actuating handle comprises the data encoding element.

38. The IVL catheter assembly of claim 36, wherein the one or more operational parameters comprise one or more catheter parameters and/or one or more balloon parameters.

39. The IVL catheter assembly of claim 36, wherein the data encoding element is configured for logging event data associated with execution of an IVL therapy via the IVL catheter assembly when the interconnect is connected to the connector receptacle.

40. The IVL catheter assembly of claim 39, wherein the event data comprises a number of voltage pulses delivered to the one or more pairs of spaced-apart electrodes when the interconnect is connected to the connector receptacle.
